(19) United States
(12) Plant Patent Application Publication
(10) Pub. No.: US 2016/0113177 P1
(43) Pub. Date: Apr. 21, 2016

(54) Anigozanthos plant named 'Carmat'

(50) ;

(71) Applicant: Grant Rankin, Hoddles Creek (AU)

(72) Inventor: Grant Rankin, Hoddles Creek (AU)

(21) Appl. No.: 14/121,817

(22) Filed: Oct. 20, 2014

(51) Int. Cl. A01H 5/00

(52) U.S. Cl. PLT/263.1

(57) ABSTRACT

A new and distinct Anigozanthos cultivar named ‘CARMAT’ is disclosed, characterized by a medium plant height, yellow flowers, well branched flowering stems of secondary ramification and light green foliage. The new variety is an Anigozanthos, normally produced as an outdoor garden or container plant.

### LATIN NAME OF THE GENUS AND SPECIES

Anigozanthos hybrid

### VARIETY DENOMINATION

‘CARMAT’

### BACKGROUND OF THE INVENTION

The new Anigozanthos cultivar is a product of chance discovery by the inventor, Grant Rankin, in Hoddles Creek, Australia. The new variety was discovered in a commercial nursery, as a single plant nutation of the commercial cultivar Anigozanthos ‘Bush Tango’, unpatented. The discovery resulting in this new variety was made during May 2009.

Asexual reproduction of the new cultivar was performed by tissue culture. This was first performed at a laboratory in Longwarry North, Australia during January 2011 and has shown that the unique features of this cultivar are stable and reproduced true to type over several successive generations.

### SUMMARY OF THE INVENTION

The cultivar ‘CARMAT’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘CARMAT’ These characteristics in combination distinguish ‘CARMAT’ as a new and distinct Anigozanthos cultivar:

1. Medium plant height
2. Secondary ramification of the inflorescence.
3. Bright yellow flowers
4. Light green foliage

### PARENT COMPARISON

Plants of the new cultivar ‘CARMAT’ are similar to plants of the parent, in most horticultural characteristics, however, plants of the new cultivar ‘CARMAT’ yellow flowers, compared to the orange-gold flowers of the parent.

### COMMERCIAL COMPARISON

Plants of the new cultivar ‘CARMAT’ are comparable to the variety Anigozanthos ‘Gold Velvet’, U.S. Plant Pat. No. 21,178. The two Anigozanthos varieties are similar in most horticultural characteristics; however, the new variety ‘CARMAT’ differs in the following:

1. Different color flowers, the comparator has gold/bronze coloration, caused by red hairs on the exterior of the perianth.
2. Different inflorescence ramification. Comparator has tertiary ramification, new variety has secondary ramification.
3. The new variety has larger flowers.

Plants of the new cultivar ‘CARMAT’ can also be compared to the commercial variety Anigozanthos ‘Bush Gold’, unpatented. These varieties are similar in most horticultural characteristics; however, the new variety ‘CARMAT’ differs in the following:

1. Different color flowers, the comparator has gold/bronze coloration, the new variety has bright yellow flowers.
2. Different inflorescence ramification. Comparator has tertiary ramification, new variety has secondary ramification.
3. The new variety has larger flowers.

### BRIEF DESCRIPTION OF THE PHOTOGRAPHS

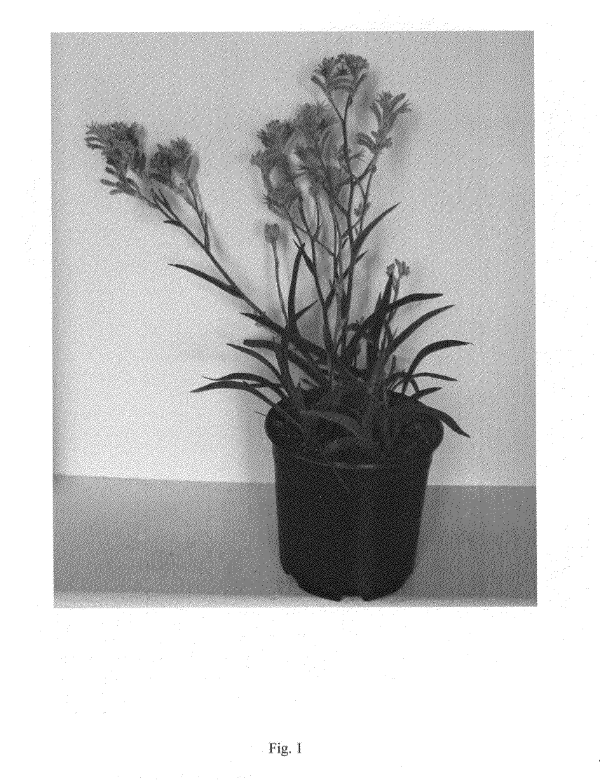

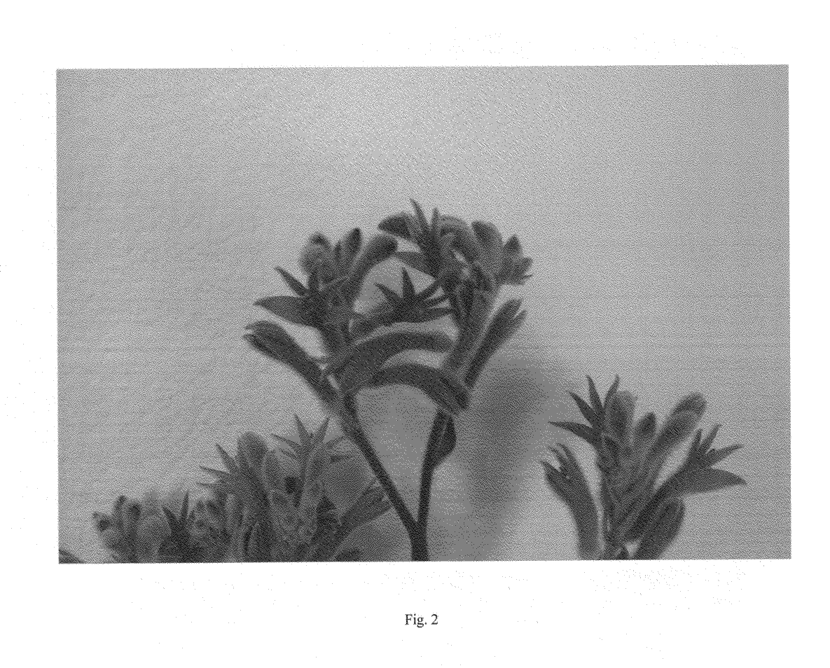

The accompanying photograph in FIG. 1 illustrates in full color a typical plant of ‘CARMAT’ grown in a greenhouse, in a 1 gallon pot. Age of the plant photographed is approximately 1 year from a rooted plantlet. FIG. 2 illustrates in full color a close up of a typical inflorescence of ‘CARMAT’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘CARMAT’ plants grown in a greenhouse, in Santa Paula, Calif. The growing temperature ranged from 20° C. to 35° C. during the day and from 17° C. to 23° C. during the night. General light conditions are bright, normal sunlight. Measurements and numerical values represent averages of typical plant types.
Botanical classification: Anigozanthos hybrid ‘CARMAT’
Propagation: Typically propagated by micropropagation tissue culture. A tissue culture plantlet is typically rooted in 1 to 2 weeks at approximately 25° C.
Root description: Root description: Thick, fleshy main roots with fibrous network of much smaller roots.
Plant:
Age of plant described: Approximately 1 year from a rooted plantlet
Growth habit: Upright fan shaped perennial with basal only branching.
Pot size of plant described: Commercial 2 gallon size container.
Height: Top of foliar plane: 20 to 25 cm.
   Top of flowering plane.—45 to 50 cm.
Plant spread: 30 to 35 cm.
Growth rate: Moderate
Foliage:
Leaf:
   Arrangement.—Basally emerging, irregular rosette. Primarily fan-shaped.
   Quantity.—Approximately 25 leaves on a 1 year old plant.
   Average Length.—Approximately 18 cm.
   Average width.—Approximately 1.6 cm(folded).
   Shape of blade.—Subulate.
   Apex.—Acuminate.
   Base.—Clasping.
   Attachment.—Sessile.
   Margin.—Entire.
   Texture of top surface.—Glabrous, margin pubescent.
   Texture of lower surface.—Glabrous, margin pubescent.
   Color.—Young foliage upper side: Near RHS Green 138A. Pubescence colored near Yellow-Orange 15C. Young foliage under side: Near RHS Green 138A. Pubescence colored near Yellow-Orange 15C. Mature foliage upper side: Near RHS Green 137A. Pubescence colored near Yellow-Orange 15C. Mature foliage under side: Near RHS Green 137A. Pubescence colored near Yellow-Orange 15C.
   Venation.—Type: Pinnate. Venation color upper side: Near RHS Green 137A. Venation color under side: Near RHS Green 137A.
   Petiole.—Not Present.
Flower:
Natural flowering season: Freely flowering under outdoors growing conditions with substantially continuous blooming from spring through autumn.
Inflorescence and flower type and habit: Large flowers arranged singly on terminal and axillary racemes. Inflorescence have secondary ramification. Individual flowers are zygomorphic, tubular, with a curved perianth.
Inflorescence height: Approximately 30 cm.
Inflorescence width: Approximately 18 cm.
Flower longevity on plant: Individual flowers last approximately 30 days.
Approximate quantity of flowers per plant: About 120 flowers and buds per plant.
Persistent or self-cleaning: Persistent.
Bud:
   Shape.—Tubular.
   Length.—About 3.5 cm.
   Diameter.—About 0.8 cm.
   Texture.—Pilous.
   Color.—Near RHS Yellow-Green 144A background coloration. Heavy pubescence colored near Yellow 13A. Apical pubescence colored near Orange-Red N34A.
Flower size:
   Diameter.—Approximately 3.0 cm.
   Length.—Approximately 4.5 cm.
Petals(unfused floral section):
   Length.—Approximately 1.2 cm.
   Diameter.—Approximately 0.4 cm.
   Quantity.—5, splitting into 6 with maturity.
   Texture.—Covered in dense, pilous hairs. Hairs colored near Yellow-Orange 17A.
   Apex.—Acute.
Color: When opening:
   Outer, visible surface.—Background color near RHS Yellow-Green 144A. Primary color from pubescence, Yellow-Orange 17A.
Fully opened:
   Outer surface.—Background color near RHS Yellow-Green 144A. Primary color from pubescence, Yellow-Orange 17A.
   Inner surface.—Near RHS Yellow-Green 144A.
Fading:
   Outer surface.—Background color near RHS Yellow-Green 144A. Primary color from pubescence, Yellow-Orange 17A.
   Inner surface.—Near RHS Yellow-Green 144A.
Perianth tube:
   Length.—Approximately 3.0 cm.
   Diameter.—Approximately 0.7 cm.
   Texture.—Outer texture highly pubescent, inner texture very slightly pubescent.
Color: When opening:
   Outer, visible surface.—Background color near RHS Yellow-Green 144A. Primary color from pubescence, Yellow-Orange 17A.
Fully opened:
   Outer surface.—Background color near RHS Yellow-Green 144A. Primary color from pubescence, Yellow-Orange 17A.
   Inner surface.—Near RHS Yellow-Green 144A.
Fading:
   Outer surface.—Background color near RHS Yellow-Green 144A. Primary color from pubescence, Yellow-Orange 17A.
   Inner surface.—Near RHS Yellow-Green 144A.
Floral bracts:
   Quantity.—1 at the base of each flower, or every other flower on inflorescence.
   Shape.—Narrow deltoid.
   Tip.—Acute.
   Base.—Clasping.
   Texture.—Upper surface glabrous, lower surface heavily pilous.
   Length.—Approximately 1.7 cm.
   Width.—Approximately 0.4 cm.
   Color.—Upper surface: Near RHS Yellow-Green 144A Lower surface: Near RHS Yellow-Green 144A. Covered in hairs near RHS Yellow 13A.
Peduncle:
   Length.—Average 8 cm.
   Diameter.—0.8 cm.
   Color.—Near RHS Yellow-Green 144A, covered in hairs colored near RHS Yellow 13A.
   Aspect.—Straight.
   Strength.—Very strong.
   Texture.—Pilous.
Pedicels:
   Length.—Average 0.5 cm.
   Diameter.—0.4 cm.
   Color.—Background color near RHS Yellow-Green 144A. Primary color from pubescence, Yellow-Orange 17A.
   Orientation.—Approximately 35 degrees from peduncle.
   Strength.—Very strong.
   Texture.—Pilous.
Sepals not present:
Fragrance: None
Reproductive organs:
Stamens:
   Number.—6.
   Filament length.—Filaments are mostly fused to petals. Approximately 0.7 cm, unfused.
   Filament color.—Near RHS Yellow-Green 144A.
Anthers:
   Shape.—Ovoid.
   Length.—Approximately 0.1 cm.
   Color.—Near RHS yellow 12A.
   Pollen.—Not observed.
Pistil:
   Number.—1.
   Length.—Approximately 2.5 cm.
   Style.—Length: Approximately 2.0 cm Color: Near RHS Yellow-Green 144B.
   Stigma.—Shape: Linear. Length: Approximately 0.1 cm. Color: Near RHS Yellow-Green 144C Ovary Color: RHS Yellow-Green 144C.
Other characteristics:
Seeds and fruits: Not observed to date.
Disease/pest resistance: Common to cultivar
Temperature tolerance: From approximately 0° C. to more than 40° C.

What is claimed is:

1. A new and distinct cultivar of Anigozanthos plant named ‘CARMAT’ as herein illustrated and described.
